(19) United States
(12) Patent Application Publication
(10) Pub. No.: US 2016/0289070 A1
(43) Pub. Date: Oct. 6, 2016

(54) PROCESSES FOR PRODUCING HYDROGEN GAS STREAM FROM AN OFFGAS STREAM

(71) Applicant: UOP LLC, Des Plaines, IL (US)

(72) Inventor: Robert S. Loboda, Hoffman Estates, IL (US)

(21) Appl. No.: 14/674,375

(22) Filed: Mar. 31, 2015

(51) Int. Cl. C01B 3/34; C07C 5/27; C10L 1/04; C10G 3/00

(52) U.S. Cl. CPC C01B 3/34; C10G 3/50; C07C 5/277; C10L 1/04; C10L 2200/0469; C10L 2290/10; C01B 2203/0216; C01B 2203/0485; C01B 2203/0425; C01B 2203/062; C01B 2203/0894; C01B 2203/0805; C01B 2203/1211; C01B 2203/1264

(57) ABSTRACT

Processes for producing a recycle hydrogen gas stream from an offgas separated from a reaction zone effluent. The reaction zone may receive a renewable feedstock and produce an effluent with gaseous components of hydrogen and light hydrocarbons. The offgas may be compressed, pass through hydrogen sulfide removal and then be sent to a reforming unit, such as a steam reformer. The steam reformer will generate additional hydrogen which can be recycled back to the reaction zone.

### FIELD OF THE INVENTION

This invention relates generally to processes which require hydrogen, and more particularly to processes which require hydrogen and which generate hydrogen from an effluent stream.

### BACKGROUND OF THE INVENTION

The use of biofuels is becoming more and more popular around the world especially based upon concerns from limited petroleum resources, increasing energy demand, greenhouse gas emissions and related climate change concerns. In addition to producing petrochemical based fuels, fuels can be manufactured using carbon and hydrogen derived from organic biomass, such as vegetable oils, organic fats, and organic greases.

For example, biological oils and fats can be converted into diesel fuel, naphtha and jet fuel using many different processes, such as hydro-deoxygenation and hydro-isomerization processes. Diesel fuel refers to a mixture of carbon chains that generally contain between 8 and 21 carbon atoms per molecule. Typically, diesel has a boiling point in the range of 180 to 380° C. (356 to 716° F.). The production of diesel fuel can be either petroleum-derived or biologically-sourced. Petroleum-derived diesel is produced from the fractional distillation of crude oil, refining products, or by conversion processes. On the other hand, biologically-sourced diesel fuel is derived from renewable feedstock, such as vegetable oils or animal fats.

The biologically-sourced diesel fuel is desirable for a variety of reasons. In addition to the ecological benefits of using biologically-sourced diesel fuel, there exists a market demand for such fuel. For diesel purchasers, the use of biologically-sourced diesel fuel can be promoted in public relations. Also, certain governmental policies may require or reward use of biologically-sourced fuels. Finally, the fluctuation of crude oil prices is also a reason refiners may choose to produce biologically-sourced fuels. The biologically-sourced diesel fuel is usually classified into two categories, biodiesel and green diesel.

Biodiesel (also referred to as fatty acid methyl ester, or FAME) mainly consists of long-chain alkyl esters typically mono-alkyl ester products derived from a lipid feed stock. The chemical structure of biodiesel is distinctly different from petroleum-derived diesel, and therefore biodiesel has somewhat different physical and chemical properties from petroleum-derived diesel. For example, biodiesel has a much higher oxygen content than petroleum-derived diesel.

Green diesel (also referred to as renewable hydrocarbon diesel, hydroprocessed vegetable oils or HVO), on the other hand, is substantially the same chemically as petroleum-derived diesel, but green diesel is made from recently living biomass. Unlike biodiesel, which is an ester and has different chemical properties from petroleum diesel, green diesel is composed of long-chain hydrocarbons, and can be mixed with petroleum diesel in any proportion for use as transportation fuel. Green diesel resembles petroleum-derived diesel fuel and usually has a very low heteroatom (nitrogen, oxygen, sulfur) content. Green diesel can thus be produced to be indistinguishable from petroleum diesel. This is beneficial because no changes to fuel infrastructure or vehicle technology are required for green diesel and it may be blended in any proportion with petroleum-derived diesel fuel as it is stable, not oxygenated.

The production of green diesel from some biomasses, such as vegetable oils, consume large amounts of hydrogen. In some areas, hydrogen is not abundantly available and therefore, reactions that require large amounts of hydrogen may be economically unviable. However, even if areas in which hydrogen is available, the required hydrogen is an added cost for a refiner. In addition to having high hydrogen demands, the decarboxylation, decarbonylation, and hydrodeoxygenation reactions associated with converting the triglycerides found in the oils into paraffins typically produce water and light hydrocarbons as well as carbon oxides.

Therefore, it would be desirable to have one or more processes that provide for an effective and efficient process to provide hydrogen for a reaction zone.

### SUMMARY OF THE INVENTION

One or more processes have been invented for the generation of a hydrogen gas stream from an offgas stream from a reaction zone. The hydrogen gas can advantageously be recycled to the reaction zone to lower the hydrogen costs associated with supplying external hydrogen for such reactions.

Therefore, in a first aspect of the invention, the present invention may be broadly characterized as a providing a process for producing a hydrogen rich gas stream by: compressing a hydrogen containing gas separated from a reaction zone in a two stage compressor system, the hydrogen containing gas including light hydrocarbons and carbon oxides; removing hydrogen sulfide from the compressed gas to provide a lean compressed gas; reforming the light hydrocarbons in the lean gas in a reforming zone to provide a hydrogen rich gas, the hydrogen rich gas including carbon oxides; and, returning the hydrogen rich gas to the reaction zone, where the carbon oxides are removed.

In one or more embodiments of the present invention, the hydrogen sulfide removal zone comprises at least one vessel comprising an adsorbent. It is contemplated that the adsorbent comprises zinc oxide. It is also contemplated that the hydrogen sulfide removal zone comprises at least two vessels comprising a zinc oxide adsorbent and the at least two vessels may be arranged in a lead-lag configuration.

In some embodiments of the present invention, the reforming zone comprises reforming with steam.

In various embodiments of the present invention, the process may comprise heating the compressed gas with the hydrogen rich gas.

In at least one embodiment of the present invention, the process may comprise heating the lean compressed gas with the hydrogen rich gas.

In one or more embodiments of the present invention, the process may comprise removing carbon oxides from the hydrogen rich gas in the reaction zone.

In various embodiments of the present invention, the reaction zone includes a deoxygenation zone for converting a renewable feedstock into hydrocarbons.

In a second aspect of the present invention, the present invention may be broadly characterized as providing a process for lowering a hydrogen demand of a user by: passing a feedstock to a deoxygenation zone to convert the feedstock into a hydrocarbon rich effluent; separating a hydrogen containing gas from the hydrocarbon rich effluent, the hydrogen containing gas including light hydrocarbons and carbon oxides; passing the hydrogen containing gas to a compression zone to provide a compressed gas; passing the compressed gas to a hydrogen sulfide removal zone configured to provide a lean compressed gas; and, passing the lean compressed gas to a reforming zone configured to convert hydrocarbons in the lean compressed gas to hydrogen and carbon oxides and provide a hydrogen rich gas.

In various embodiments of the present invention, the process may comprise passing the hydrocarbon rich effluent to an isomerization zone to convert linear hydrocarbons to branched hydrocarbons and provide an isomerized effluent. The hydrogen containing gas may be separated from the isomerized effluent. It is contemplated that the process comprises removing carbon oxides from a recycle gas to the deoxygenation zone in a purification zone.

In at least one embodiments of the present invention, the purification zone removes carbon dioxide by amine scrubbing.

In one or more embodiments of the present invention, the hydrogen sulfide removal zone comprises two vessels arranged in a lead-lag configuration and each vessel comprising a zinc oxide adsorbent.

In some embodiments of the present invention, the compression zone comprises a two stage compression zone.

In various embodiments of the present invention, the reforming zone comprises a steam reforming zone, and the process further comprises cooling the hydrogen rich gas by generating steam. It is contemplated that the process includes heating at least one of the compressed gas or the lean compressed gas with the hydrogen rich gas. It is also contemplated that the process includes heating both the compressed gas and the lean compressed gas with the hydrogen rich gas.

In at least one embodiment of the present invention, the feedstock is a renewable feedstock.

In one or more embodiments of the present invention, the process may comprise combining the hydrogen rich gas with a stream from the deoxygenation zone.

Additional aspects, embodiments, and details of the invention, which may be combined in any manner, are set forth in the following detailed description of the invention.

### DETAILED DESCRIPTION OF THE DRAWINGS

One or more exemplary embodiments of the present invention will be described below in conjunction with the following drawing figures, in which:

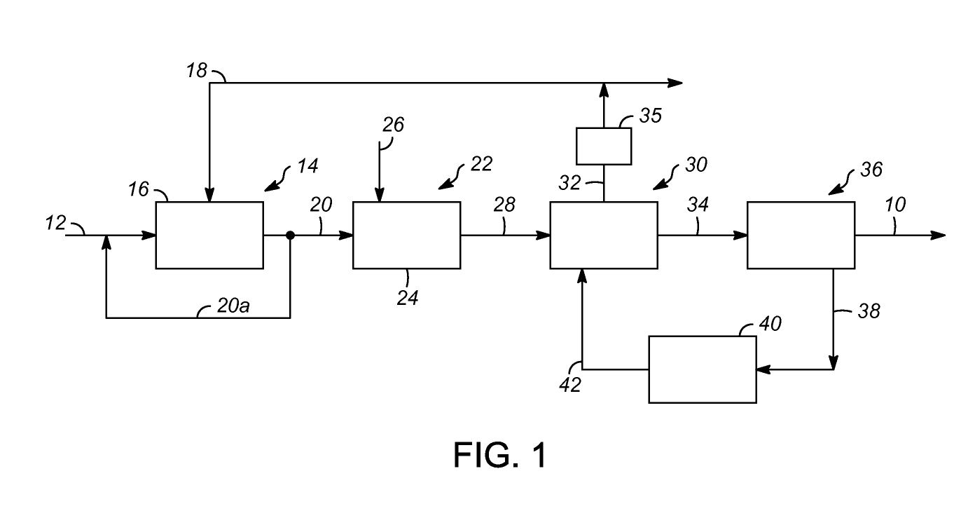

FIG. 1 shows a process flow diagram according to one or more embodiments of the present invention; and,

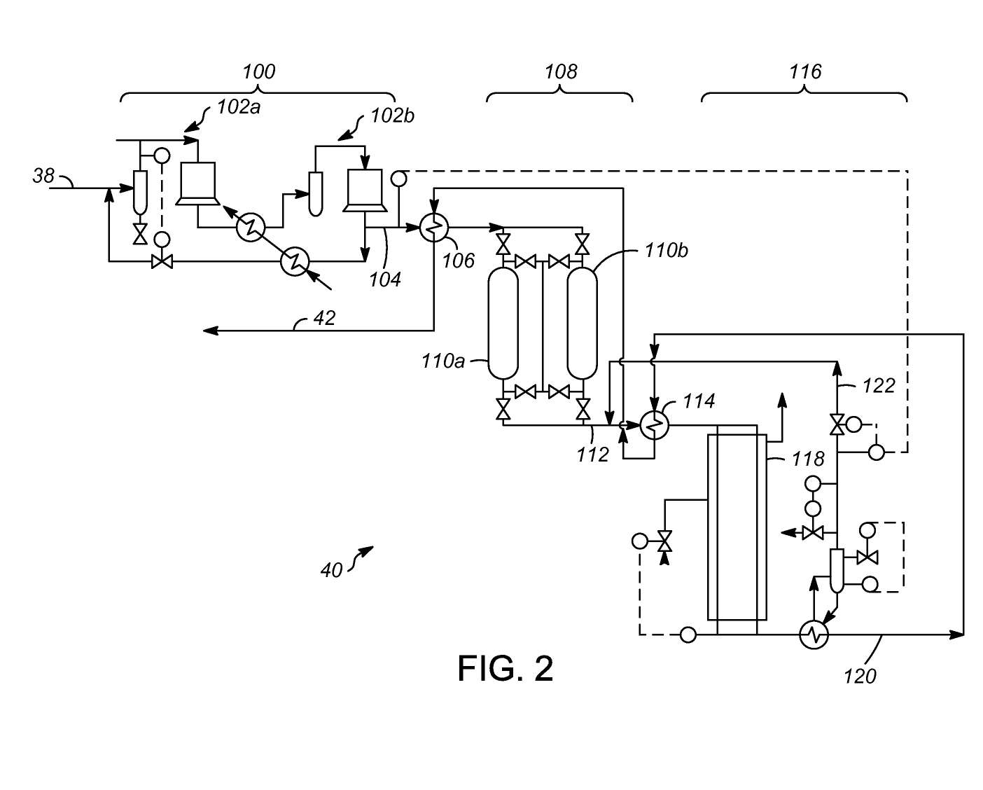

FIG. 2 shows another process flow diagram according to one or more embodiments of the present invention.

### DETAILED DESCRIPTION OF THE INVENTION

As mentioned above, one or more processes have been invented in which a gas stream that is usually used as fuel can be used as hydrogen gas. A reformer may increase the amount of hydrogen gas. It is believed that the processes could reduce the amount of hydrogen purchased by as much as 50%. In addition to lowering the hydrogen demand, certain equipment, such as an offgas sponge absorber could be eliminated. While additional equipment and operating utilities associated for the equipment would result in an increase, the savings from the hydrogen demand, especially in areas where hydrogen is expensive, are believed to offset these capital and utility expenditures. Thus, the utilization of an offgas stream for production hydrogen to recycle back to a reaction zone is believed to be desirable.

With these general principles in mind, one or more embodiments of the present invention will be described with the understanding that the following description is not intended to be limiting.

Turning to FIG. 1, the various embodiments of the present invention relate to a process for producing a hydrocarbon stream 10, useful as diesel boiling range fuel for example, from a renewable feedstock 12 such as renewable feedstocks originating from plants or animals. Some of these renewable feedstocks are known as biorenewable fats and oils. The term “renewable feedstock” is meant to include feedstocks other than those obtained from crude oil. The renewable feedstock 12 may include any of those feedstocks which comprise at least one of glycerides and free fatty acids (FFA). Most of the glycerides will be triglycerides, but monoglycerides and diglycerides may be present and processed as well. Examples of these renewable feedstocks 12 include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm oil, mustard oil, tallow, yellow and brown greases, lard, train oil, fats in milk, fish oil, algal oil, sewage sludge, and the like. Additional examples of renewable feedstocks 12 include non-edible vegetable oils from the group comprising Jatropha curcas (Ratanjot, Wild Castor, Jangli Erandi), Madhuca indica (Mohuwa), Pongamia pinnata (Karanji, Honge), calophyllum inophyllum, moringa oleifera and Azadirachta indica (Neem). The triglycerides and FFAs of the typical vegetable or animal fat contain aliphatic hydrocarbon chains in their structure which have about 8 to about 30 carbon atoms.

The renewable feedstock 12 may contain a variety of impurities. For example, tall oil is a byproduct of the wood processing industry and tall oil contains esters and rosin acids in addition to FFAs. Rosin acids are cyclic carboxylic acids. The renewable feedstocks 12 may also contain contaminants such as alkali metals, e.g. sodium and potassium, phosphorous as well as solids, water and detergents. Therefore, an optional first step is to remove as much of these contaminants as possible in a pretreatment zone (not shown) to provide a pretreated feedstock. Various pretreatment zones are known in the art.

Returning to FIG. 1, the renewable feedstock 12 may be converted to hydrocarbons in a deoxygenation zone 14 comprising one or more catalyst beds in one or more reactors 16. More specifically, in the deoxygenation zone 14, the renewable feedstock 12 is contacted with a catalyst in the presence of hydrogen at deoxygenation conditions to deoxygenate the renewable feedstock 12. Deoxygenation reactions, including decarbonylation, decarboxylation, hydrodeoxygenation and deoxygenation, result in oxygen being removed. In order to provide hydrogen for these reactions, the deoxygenation zone 14 receives a hydrogen containing gas 18. In addition to deoxygenation, olefinic, or unsaturated, portions of the hydrocarbons may be hydrogenated in the deoxygenation zone 14.

Suitable catalysts and conditions for the deoxygenation reaction zone are known in the art, and are disclosed in for example U.S. Pat. No. 8,865,954, the entirety of which is incorporated herein by reference. For example, the deoxygenation zone 14 may include nickel or nickel/molybdenum dispersed on a high surface area support. Generally, deoxygenation conditions include a temperature of about 40 to about 700° C. (104 to 1292° F.) and a pressure of about 700 kPa (100 psig) to about 21 MPa (3000 psig).

In some embodiments, for example those in which the hydrocarbon stream 10 comprises a diesel product stream, the deoxygenated effluent 20 may be passed from the deoxygenation zone 14 to an isomerization zone 22. Although not depicted as such, it is contemplated that the deoxygenation zone 14 and the isomerization zone 22 are contained within the same reactor vessel. Additionally, a portion 20a of the deoxygenated effluent 20 may be recycled to the deoxygenation zone 14. Additionally, a high purity hydrogen containing gas 26 is also passed to the isomerization zone 22.

The isomerization of the normal hydrocarbons in a reactor 24 in the isomerization zone 22 can be accomplished in any manner known in the art or by using any suitable catalyst known in the art. One or more beds of catalyst may be used and the isomerization may be operated in a co-current mode of operation. Fixed bed, trickle bed down flow or fixed bed liquid filled up-flow modes are both suitable. Suitable catalysts may comprise a metal of Group VIII (IUPAC 8-10) of the Periodic Table and a support material and are described in detail in U.S. Pat. No. 8,865,593, the entirety of which is incorporated herein.

An isomerized effluent 28 from the isomerization zone 22 comprises a branched-paraffin-rich stream. By the term “rich” it is meant that the effluent stream has a greater concentration of branched paraffins than the stream entering the isomerization zone 22, and preferably comprises greater than 50 mass-% branched paraffins. It is envisioned that the isomerized effluent 28 may contain 70, 80, or 90 mass-% branched paraffins. For producing a diesel product stream, only minimal branching is required, enough to overcome the cold-flow problems of the normal paraffins. Since attempting for significant branching runs the risk of a high degree of undesired cracking, the predominant product in the isomerized effluent 28 is a mono-branched paraffin.

From the isomerization zone 22, the isomerized effluent 28, or if no isomerization zone 22 is utilized, the deoxygenated effluent 20 (or a liquid portion of the deoxygenated effluent 20), is passed to a separation zone 30.

The separation zone 30 may include any equipment capable of separating the isomerized effluent 28 into various components. In one embodiment (not shown), the separation zone 30 comprises at least a cooler, a cold product separator, and a product recovery column. In any configuration, the separation zone 30 may provide a gaseous stream 32 and a liquid stream 34.

The gaseous stream 32 comprises carbon oxides, hydrogen sulfide, and hydrogen. Stream 32 may be passed to a scrubbing (or purification) zone 35 having one or more selective amine solution absorbers configured to remove carbon dioxide. Most of the scrubbed hydrogen gas comprises the hydrogen containing gas 18 and is recycled to the deoxygenation zone 14. However, some of the hydrogen containing gas leaves the process in order to limit residual methane and carbon monoxide.

The liquid stream 34 from the separation zone 30 is passed to a product recovery zone 36 to separate the components of the liquid stream 34 into the hydrocarbon stream 10 and an offgas stream 38. The hydrocarbon stream 10 comprises the desired product, for example a diesel range fuel stream. The offgas stream 38 comprises hydrogen, light hydrocarbons (i.e., C₅-hydrocarbons), carbon oxides and hydrogen sulfide. Typically the offgas stream 38 must be scrubbed to remove hydrogen sulfide before being used a fuel for a heater. However, since the offgas stream 38 contains hydrogen, carbon monoxide and light hydrocarbons which can be converted to hydrogen, the processes according to the present invention utilizes a reforming zone 40 to provide a compressed recycle hydrogen gas stream and reform it, thus generating additional hydrogen contained in stream 42. A preferred reforming zone 40 is depicted in FIG. 2.

As shown in FIG. 2, the reforming zone 40 comprises a compression zone 100, which is preferably comprises a two stage compression with two compressors 102a, 102b. The offgas stream 38 is compressed in the compression zone 100, by increasing the pressure as much as 14 times the pressure of the offgas stream 38. A compressed offgas gas 104 is passed from the compression zone 100, heated in a heat exchange zone 106, and passed to a hydrogen sulfide removal zone 108.

The hydrogen sulfide removal zone 108 may include one or more vessels 110a, 110b having an adsorbent capable of selectively adsorbing hydrogen sulfide from the compressed offgas gas 104 to provide a lean compressed gas 112. Preferably, the hydrogen sulfide removal zone 108 comprises two vessels 110a, 110b arranged in a lead-lag configuration. Upon saturation, or near saturation, of the adsorbent in the first vessel 110a, that vessel is bypassed and isolated for a changeout of adsorbent. When the second vessel 110b has become saturated, the first vessel 110a with fresh adsorbent can be utilized in a similar manner.

The lean compressed gas 112 from the hydrogen sulfide removal zone 108 may be heated in a heat exchange zone 114 and passed to a reformer unit 116. In a preferred embodiment, the reformer unit 116 comprises a steam reformer 118 in which high pressure steam will react with the light hydrocarbons in the lean compressed gas 112 to form hydrogen and carbon oxides in a hydrogen rich gas stream 120. As will be appreciated, the steam reformer unit 116 may include a process gas boiler which provides a steam stream 122 for the steam reformer 118 as well as some net export.

The hydrogen rich gas stream 120 may pass through the heat exchange zone 114 to heat the lean compressed gas 112 and then to the heat exchange zone 106 to heat the compressed offgas gas 104. From there, the hydrogen rich gas stream 120 can be returned to one or more reaction zones as the compressed recycle hydrogen gas stream 42.

Returning to FIG. 1, the compressed recycle hydrogen gas stream 42 may be passed to the separation zone 30 to be separated and returned to the deoxygenation zone 14.

It is contemplated, although not depicted, that the reforming zone 40 includes a shift reactor to improve the recycle gas purity by converting carbon monoxide to scrubable carbon dioxide.

By compressing, scrubbing and creating additional hydrogen from an offgas stream that is typically used as less valuable fuel gas, the hydrogen demands of a user, such as a refiner or plant operator may be lowered.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing a hydrogen rich gas stream, the process comprising:
compressing a hydrogen containing gas separated from a reaction zone in a two stage compressor system, the hydrogen containing gas including light hydrocarbons and carbon oxides;
removing hydrogen sulfide from the compressed gas in a hydrogen sulfide removal zone to provide a lean compressed gas;
reforming the light hydrocarbons in the lean gas in a reforming zone to provide a hydrogen rich gas, the hydrogen rich gas including carbon oxides; and,
returning the hydrogen rich gas to the reaction zone.

2. The process of claim 1 wherein the hydrogen sulfide (removal zone comprises at least one vessel comprising an adsorbent.

3. The process of claim 2 wherein the adsorbent comprises zinc oxide.

4. The process of claim 3 wherein the hydrogen sulfide removal zone comprises at least two vessels comprising a zinc oxide adsorbent, the at least two vessels being arranged in a lead-lag configuration.

5. The process of claim 1 wherein the reforming zone comprises reforming with steam.

6. The process of claim 1 further comprising:
heating the compressed gas with the hydrogen rich gas.

7. The process of claim 1 further comprising:
heating the lean compressed gas with the hydrogen rich gas.

8. The process of claim 1 further comprising:
removing carbon oxides from the hydrogen rich gas in the reaction zone.

9. The process of claim 1 wherein the reaction zone includes a deoxygenation zone for converting a renewable feedstock into hydrocarbons.

10. A process for lowering a hydrogen demand of a user, the processing comprising:
passing a feedstock to a deoxygenation zone to convert the feedstock into a hydrocarbon rich effluent;
separating a hydrogen containing gas from the hydrocarbon rich effluent, the hydrogen containing gas including light hydrocarbons and carbon oxides;
passing the hydrogen containing gas to a compression zone to provide a compressed gas;
passing the compressed gas to a hydrogen sulfide removal zone configured to provide a lean compressed gas; and,
passing the lean compressed gas to a reforming zone configured to convert hydrocarbons in the lean compressed gas to hydrogen and carbon oxides and provide a hydrogen rich gas.

11. The process of claim 10 further comprising:
passing the hydrocarbon rich effluent to an isomerization zone to convert linear hydrocarbons to branched hydrocarbons and provide an isomerized effluent, wherein the hydrogen containing gas is separated from the isomerized effluent.

12. The process of claim 11 further comprising:
removing carbon oxides from a recycle hydrogen gas passed to the deoxygenation zone in a purification zone.

13. The process of claim 12 wherein the purification zone includes carbon dioxide removal by amine scrubbing.

14. The process of claim 10 wherein the hydrogen sulfide removal zone comprises two vessels arranged in a lead-lag configuration and each vessel comprising a zinc oxide adsorbent.

15. The process of claim 10 wherein the compression zone comprises a two stage compression zone.

16. The process of claim 10 wherein the reforming zone comprises a steam reforming zone, and wherein the process further comprises:
cooling the hydrogen rich gas by generating steam.

17. The process of claim 16 further comprising:
heating at least one of the compressed gas or the lean compressed gas with the hydrogen rich gas.

18. The process of claim 17 further comprising:
heating both the compressed gas and the lean compressed gas with the hydrogen rich gas.

19. The process of claim 10 wherein the feedstock is a renewable feedstock.

20. The process of claim 10 further comprising combining the hydrogen rich gas with a stream from the deoxygenation zone.
